(19) United States
(12) Patent Application Publication
(10) Pub. No.: US 2013/0345649 A1
(43) Pub. Date: Dec. 26, 2013

(54) Self-Heating Patch

(71) Applicant: Sealed Air Corporation(US), Duncan, SC (US)

(72) Inventors: Henry Walker Stockley, III, Spartanburg, SC (US); William Peyton Roberts, Spartanburg, SC (US); Stephen F. Compton, Spartanburg, SC (US); Drew Ve Speer, Simpsonville, SC (US)

(73) Assignee: Sealed Air Corporation(US), Duncan, SC (US)

(21) Appl. No.: 13/905,234

(22) Filed: May 30, 2013

Related U.S. Application Data
(60) Provisional application No. 61/697,337, filed on Sep. 6, 2012.
(60) Provisional application No. 61/664,323, filed on Jun. 26, 2012.

(51) Int. Cl. A61F 7/03; A61K 9/70

(52) U.S. Cl. CPC A61F 7/034; A61K 9/7084
USPC 604/304; 607/114; 156/250; 156/196

(57) ABSTRACT

A self-heating patch includes a bottom barrier layer, an air-activated heat-generating layer, an air regulation layer, optionally a top barrier layer, and optionally a skin contact layer, wherein the air-activated heat-generating layer is encapsulated by the air regulation layer and bottom barrier layer, and/or the top and bottom barrier layers, and the temperature of the patch, when the air-activated heat-generating layer has been exposed to air, and when the top barrier layer if present is removed, is controlled at least in part by the air regulation layer; and a perimeter seal seals the air regulation layer to the bottom barrier layer around the perimeter of the patch. Optionally a temperature-responsive mechanism, such as a wax, can be used to reduce oxygen intake into the self-heating patch and thereby control the temperature of the self-heating patch during use. Optionally, a portion of the self-heating patch can be thermoformed.

This application claims the benefit of U.S. Provisional Application No. 61/664,323 filed Jun. 26, 2012, and U.S. Provisional Application No. 61/697,337 filed Sep. 6, 2012, these applications incorporated herein by reference in their entirety.

### FIELD OF THE INVENTION

This invention relates to a self-heating patch, and to a method of making the self-heating patch.

### BACKGROUND OF THE INVENTION

Dermal patches, such as back warmers, face masks, hand warmers, and thermal wraps for placement on the body to heat muscles, are typically made with air activated exothermic reactive materials, such as salt-activated iron powders, that achieve temperature regulation by means of a formulation having a low energy density and reaction rate, such that their own heat capacity and reaction rate limitations serve to regulate the temperature rise. They are unfortunately not amenable to use as relatively thin, lightweight skin patches.

Other currently available heat generating sachets employ a substance, such as calcium oxide, that will react with water to generate heat. These produce steam, however, and must be vented. Further, they are difficult to control and may be subject to over-heating in skin-contact applications.

Some heaters employ a liquid-solid phase change material to deliver heat at a well regulated temperature. However, the phase change material is not itself a heat generator unless pre-heated by some other means. Also, such heaters tend to be bulky and heavy. Further, use of a phase change material requires a more complex structure with separate compartments for heat generation and phase change materials respectively.

There is need in the marketplace for a relatively thin, light patch that delivers a sufficient amount of heat for a sufficient duration and yet does not overheat; and that does not require water, or a liquid-solid phase change, to generate heat.

There is also a need in the marketplace for a relatively thin, light patch that delivers heat as well as an active ingredient, e.g. a therapeutic or cosmetic agent, to the skin surface. Currently available hand warmers, body warmers, etc. are not well suited to the delivery of an active ingredient to the skin surface. These packs contain loose material that can shift around, making them susceptible to uneven distribution of heat when used in a thin patch. Also, the containment construction typically involves a thick nonwoven material on all sides of the pack. If used in conjunction with skin treatments such as wrinkle cream, the cream would be absorbed by the non-woven, wasting it and allowing it to contact the heat-generating material inside.

### SUMMARY OF THE INVENTION

In a first aspect, a flexible, self-heating patch comprises:

a) a bottom barrier layer;

b) an air-activated heat-generating layer; and

c) an air regulation layer;

wherein the air-activated heat-generating layer is encapsulated by the air regulation layer and the bottom barrier layer;

wherein the temperature of the patch, when the air-activated heat-generating layer has been exposed to air, is controlled at least in part by the air regulation layer; and

wherein a perimeter seal seals the air regulation layer to the bottom barrier layer around the perimeter of the patch.

In a second aspect, a method of making a plurality of self-heating patches comprises

a) providing an air regulation layer;

b) applying an air-activated heat-generating layer to the air regulation layer to form a top sub-assembly as a first web;

c) providing a bottom sub-assembly comprising a bottom barrier layer and a skin contact layer to form a second web;

d) applying an activation agent to the air-activated heat-generating layer to change the air-activated heat-generating layer from an air-stable state to an air-reactive state;

e) bringing the first and second webs together to encapsulate the air-activated heat-generating layer; and

f) cutting and sealing the first and second webs to form a plurality of self-heating patches each with a perimeter seal.

In a third aspect, a method of making a plurality of self-heating patches comprises

a) providing a bottom sub-assembly comprising a bottom barrier layer and a skin contact layer as a first web;

b) applying an air-activated heat-generating layer to the bottom sub-assembly;

c) providing an air regulation layer as a second web;

d) applying an activation agent to the air-activated heat-generating layer to change the air-activated heat-generating layer from an air-stable state to an air-reactive state;

e) bringing the first and second webs together to encapsulate the air-activated heat-generating layer; and

f) cutting and sealing the first and second webs to form a plurality of self-heating patches each with a perimeter seal.

In a fourth aspect, a method of making a plurality of self-heating patches comprises

a) providing a peelable composite comprising a top barrier layer and an air regulation layer;

b) applying an air-activated heat-generating layer to the air regulation layer to form a top sub-assembly as a first web;

c) providing a bottom sub-assembly comprising a bottom barrier layer and a skin contact layer to form a second web;

d) applying an activation agent to the air-activated heat-generating layer to change the air-activated heat-generating layer from an air-stable state to an air-reactive state;

e) bringing the first and second webs together to encapsulate the air-activated heat-generating layer, and

f) cutting and sealing the first and second webs to form a plurality of self-heating patches each with a perimeter seal.

In a fifth aspect, a method of making a plurality of self-heating patches comprises

a) providing a bottom sub-assembly comprising a bottom barrier layer and a skin contact layer as a first web;

b) applying an air-activated heat-generating layer to the bottom sub-assembly;

c) providing a peelable composite comprising a top barrier layer and an air regulation layer as a second web;

d) applying an activation agent to the air-activated heat-generating layer to change the air-activated heat-generating layer from an air-stable state to an air-reactive state;

e) bringing the first and second webs together to encapsulate the air-activated heat-generating layer, and

f) cutting and sealing the first and second webs to form a plurality of self-heating patches each with a perimeter seal.

In a sixth aspect, a flexible, self-heating patch comprises:

a) a first segment comprising
i) a thermoformed peelable composite comprising a barrier layer and an air regulation layer; and
ii) an air-activated heat-generating layer; and

b) a second segment comprising an interface film;

wherein the air-activated heat-generating layer is encapsulated by the air regulation layer and the interface film;

wherein the temperature of the patch, when the air-activated heat-generating layer has been exposed to air, is controlled at least in part by the air regulation layer; and

wherein a perimeter seal seals the first segment to the second segment around the perimeter of the patch.

In a seventh aspect, a method of making a self-heating patch comprises
a) providing a peelable composite comprising a barrier layer and an air regulation layer;
b) thermoforming the peelable composite to form a pocket;
c) applying an activation agent to the pocket;
d) applying an air-activated heat-generating layer to the pocket;
wherein the peelable composite, activation agent, and air-activated heat-generating layer comprise a first segment;
e) applying an interface film to the first segment to encapsulate the air-activated heat-generating layer between the first segment and the interface film; and
f) sealing the perimeter of the first and second segments to form a self-heating patch with a perimeter seal.

### BRIEF DESCRIPTION OF THE DRAWINGS

The present invention is illustrated by reference to the following drawings, encompassing different views of various embodiments of the invention, wherein:

FIG. 1 is a cross-sectional view of a self-heating patch;

FIG. 2 is a cross-sectional view of a self-heating patch in accordance with an alternative embodiment of the invention;

FIG. 3 is a cross-sectional view of a top sub-assembly for use with the invention;

FIG. 4 is a cross-sectional view of a bottom sub-assembly for use with the invention;

FIG. 5 is a cross-sectional view of a peelable web for use with the invention;

FIG. 6 is a cross-sectional view of the peelable web of FIG. 5, as a first portion of the peelable web is being peeled away;

FIG. 7 is a cross-sectional view of another embodiment of a bottom sub-assembly for use with the invention;

FIG. 8 is a plan view of a self-heating patch;

FIG. 9 is a schematic view of an apparatus and process for making a self-heating patch;

FIG. 10 is a schematic view of an alternative apparatus and process for making a self-heating patch;

FIG. 11 is a plan view of an air-activated heat-generating layer of the self-heating patch, in an alternative embodiment;

FIG. 12 is a cross-sectional view of a portion of the self-heating patch, in another embodiment;

FIG. 13 is an enlarged view of a portion of FIG. 12;

FIG. 14 is a cross-sectional view of a portion of the self-heating patch, in still another embodiment;

FIG. 15 is a cross-sectional view of a portion of the self-heating patch, in yet another embodiment,

FIG. 16 is a cross-sectional view of a peelable composite for use with the invention;

FIG. 17 is a cross-sectional view of the peelable composite of FIG. 16 after it has been thermoformed;

FIG. 18 is a cross-sectional view of the thermoformed peelable composite of FIG. 17 after an activation agent has been applied;

FIG. 19 is a cross-sectional view of the thermoformed peelable composite of FIG. 18 after an air distribution layer has been applied;

FIG. 20 is a cross-sectional view of the thermoformed peelable composite of FIG. 19 after an air-activated heat-generating layer has been applied; and

FIG. 21 is a cross-sectional view of the thermoformed peelable composite of FIG. 20 after an interface film has been applied;

### DEFINITIONS

“Activation agent” herein refers to any suitable agent, such as an aqueous electrolyte such as a concentrated KOH solution, that changes the air-activated heat-generating layer from an air-stable state to an air-reactive state.

“Film” is used herein to mean a film, laminate, or web, either multilayer or monolayer, that may be used in connection with the present invention.

“Flexible” herein refers to a self-heating patch capable of substantially conforming to the surface of the skin.

“Frit” herein refers to a porous member that allows gases to migrate through the frit material, but does not allow substantial migration of solids or liquids through the frit material. Examples include polytetrafluoroethylene (PTFE) powder, or the fused or partially fused materials, including silica and fluxing agents, used in making glass.

“Oxygen barrier” and the like herein refers to materials having an oxygen permeability, of the barrier material, less than 500 cm³ O₂/m²·day·atmosphere (tested at 1 mil thick and at 25° C., 0% RH according to ASTM D3985), such as less than 100, less than 50, less than 25, less than 10, less than 5, and less than 1 cm³ O₂/m²·day·atmosphere. Examples of polymeric materials useful as oxygen barrier materials are ethylene/vinyl alcohol copolymer (EVOH), polyvinylidene dichloride (PVDC), vinylidene chloride/methyl acrylate copolymer, vinylidene chloride/vinyl chloride copolymer, polyamide, and polyester. Examples of polymeric materials having an oxygen permeability, of the barrier material, less than 50 cm³ O₂/m²·day·atmosphere are ethylene/vinyl alcohol copolymer (EVOH), polyvinylidene dichloride (PVDC), vinylidene chloride/methyl acrylate copolymer, and vinylidene chloride/vinyl chloride copolymer.

“Layer” herein refers, in the sense of a component of the patch of the invention, to a monolayer or multilayer coating, or to a monolayer or multilayer film structure produced by any suitable process, such as coextruded, lamination, extrusion coating, extrusion lamination, printing, and the like.

“Therapeutic” and the like herein means:

1) a drug, i.e. an article intended for use in the diagnosis, cure, mitigation, treatment, or prevention of disease, or an article, other than food, intended to affect the structure or any function of the body, and/or

2) a cosmetic, i.e. an article intended to be introduced into, or otherwise applied to the human body, for cleansing, beautifying, promoting attractiveness, or altering the appearance. Examples include skin moisturizer, eye and facial makeup preparations, and the like.

All compositional percentages used herein are presented on a “by weight” basis, unless designated otherwise.

### DETAILED DESCRIPTION OF THE INVENTION

The invention is directed to a relatively thin (e.g. 0.5 to 3 mm thick) and flexible self-heating patch, adapted for skin contact applications. The function of this patch, when activated by the user and applied to a skin surface, is to transmit heat, and optionally a therapeutic agent, to the skin surface in order to afford warmth and/or to enhance the effectiveness of a therapeutic agent such as e.g. wrinkle cream. The patch adheres removably to the skin and functions over any suitable amount of time, e.g. ranging from about 5 minutes to about 8 hours. The patch comprises an air-activated heat-generating layer surrounded by film layers that perform various functions. These layers are disclosed in further detail below, and in the drawings. Some of these features are optional and not present in all embodiments.

Skin Contact Layer

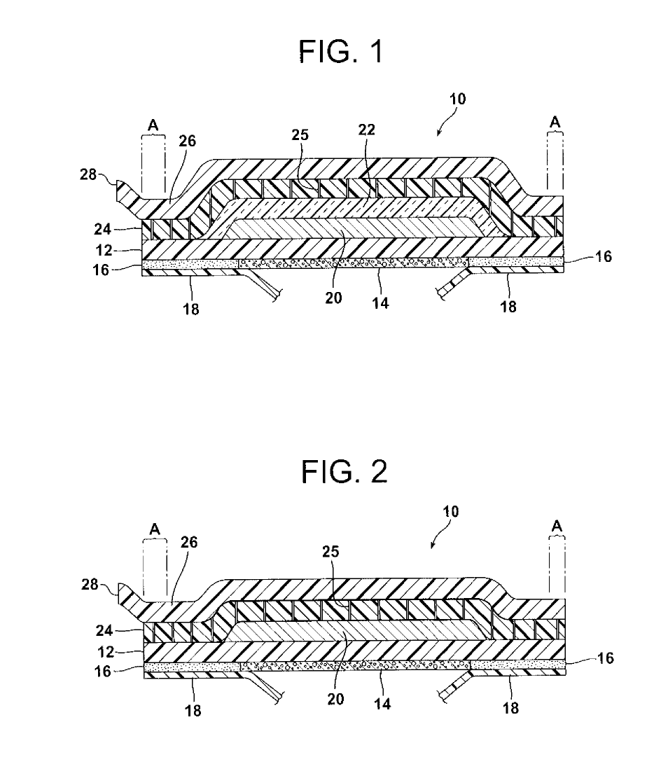

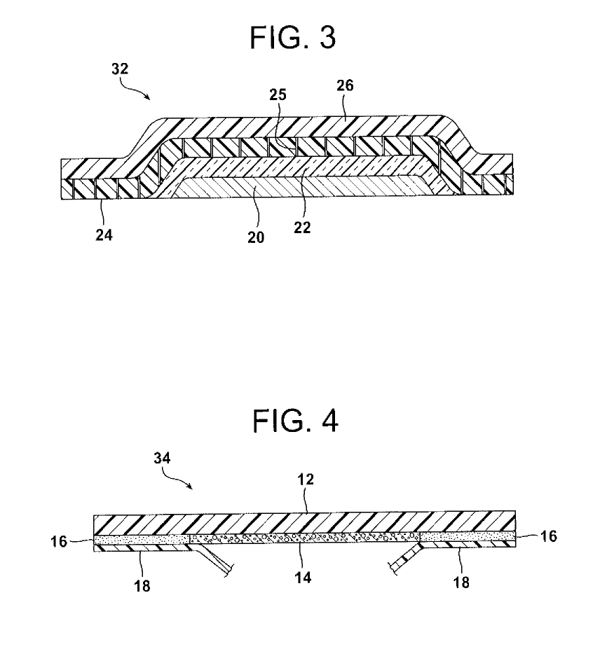

Referring to FIG. 1, the underside of the self-heating patch 10 comprises a skin contact layer 14 positioned towards the center of the patch and intermediate the edges of the patch. The function of the skin contact layer 14 is to present a soft, comfortable surface, and in some embodiments to provide a matrix for holding and releasing a therapeutic agent to the skin surface during use. Any suitable material can be used for skin contact layer 14, such as nonwoven fabric or open-celled foam sheet.

Adhesive Layers

Adhesive layers 16 are positioned as shown in FIG. 1 towards opposing ends or edges of patch 10, and can in one embodiment extend under the skin contact layer 14 as well. In this latter embodiment, one adhesive layer 16 is present. Any conventional adhesive, such as pressure sensitive adhesive, can be used. Adhesive layers 16 serve to removably adhere the patch 10 to the user's skin.

Peelable Adhesive Liners

The adhesive layers and skin contact layer can in one embodiment be covered by one or more peelable adhesive liners 18. These can be made from a silicone-coated paper or other suitable material, and used to prevent contamination of the adhesive of the adhesive layers until the patch is to be adhered to the skin of a user. At such time as the patch is to be used, the adhesive liners are peeled away from the adhesive, exposing the adhesive so that it can be used to adhere the patch 10 to the user's skin. In one embodiment, a single peelable adhesive liner extends across the skin contact layer 14 as well as the adhesive layers 16.

Bottom Barrier Layer

The bottom barrier layer 12 is a flexible film that affords a continuous substrate to directly or indirectly support the other layers of the patch 10. Bottom barrier layer 12 functions to prevent oxygen exterior to the patch 10 from reaching the interior of the patch, and in particular from prematurely reaching the heat-generating layer. The oxygen transmission rate of the bottom barrier layer 12 must be less than about 50,000 cc/m²/day/atm so as to prevent undesired oxygen-induced heating of the skin patch prior to or during its use. The oxygen transmission rate of the bottom barrier layer 12 should be less than 500 cc/m²/day/atm unless the patch is stored in an outer barrier package prior to use, to ensure that oxygen absorption capacity of the heating layer is not gradually depleted prior to use. Any suitable film of any appropriate thickness can be used for bottom barrier layer 12. The bottom barrier layer may comprise any polymeric composition provided at least one layer is in a continuous film form. It may be especially desirable for the bottom barrier layer 12 to be a high oxygen barrier film, in which case it comprises an oxygen barrier material of any suitable kind, organic or inorganic in nature, including one or more of the polymeric materials identified herein.

Disclosed herein are examples of polymeric materials useful as oxygen barrier materials. Besides its function as a supporting substrate, and as a barrier to oxygen, bottom barrier layer 12 provides a surface to which the air regulation layer, to be discussed in more detail below, can be sealed. This sealing can be accomplished by making a perimeter seal around the edges of the patch, making any suitable type of seal such as a heat seal, ultrasonic seal, radio frequency seal, adhesive seal, or the like. When used in conjunction with a top barrier layer, to be discussed in more detail below, the perimeter seal provides an initially hermetic patch 10.

Heat-Generating Layer

The heat-generating layer 20 comprises an oxidizable metal (e.g., magnesium, zinc, aluminum, iron, etc.), an oxidation accelerator (e.g. carbon black, salt, and water), and a binder (e.g. polytetrafluoroethylene (PTFE), wax, polyethylene). When activated by an activation agent, if required, and exposed to air, this layer undergoes a spontaneous and highly exothermic oxidation reaction to generate heat and metal oxide byproducts. Air activated heat-generating layers and formulations are well known. An example is the material produced by Rechargeable Battery Corporation, College Station, Tex., (RBC), currently used inside COOK PAK™ heater packs designed for use with food such as military rations. The RBC material has good flexibility, cohesion, energy density and heat generation rate.

In one embodiment, the heat-generating layer may comprise a temperature-responsive mechanism such as a low-melting component, such as a paraffin wax powder. In the solid state, this wax powder supports a high level of internal porosity within the heating layer. However, in the event that the heat-generating layer reaches an excessively high temperature, the wax will melt and, in so doing substantially reduce internal porosity to curtail internal air flow within the heat-generating layer, and/or the molten wax will form a coating on the oxygen-reactive carbon surfaces within the heater material to block further reactions from taking place. By one or more of these mechanisms, the wax will serve as an overheat-protection means. Suitable waxes include hydrocarbon waxes such as paraffin, montan, micro crystalline waxes, polyolefin waxes (PE, PP, EVA), natural waxes such as carnauba wax, candelilla, beeswax and the like, fatty acids, amide waxes, hydrogenated vegetable oils and mixtures thereof.

In one embodiment, the heat-generating layer 20 is centrally disposed within the patch, such that the presence of the bottom barrier layer 12, and top barrier layer 26, along with the perimeter seal 30 (see FIG. 8), assure that the heat-generating layer does not prematurely activate, or continue its activity if previously initiated, while within the hermetic patch. In one embodiment, for the sake of efficiency, the heat-generating layer 20 is positioned so as to substantially coincide with the lateral extent of the skin contact layer 14. This relationship is shown in FIGS. 1 and 2.

Air Distribution Layer

The next layer is the air distribution layer 22. This can comprise any suitable material, e.g. one that has a highly textured surface on both sides and/or a high level of porosity. The primary function of this layer is to facilitate the movement and distribution of air from the exterior of the patch 10 to the adjacent surface of the heat-generating layer, to functionally support the oxidation reaction within the heat-generating layer. The air distribution layer can comprise, for example, a nonwoven or loosely woven mat, a loosely compacted set of particles like a frit, an open celled foam, or a perforated and embossed film. A textured surface could also be created through printing, e.g., a foaming ink.

In some embodiments, the air distribution layer provides some thermal insulation, thereby limiting heat loss from the patch to the air and maximizing the amount of heat transferred to the skin.

Alternatively (see FIG. 2), the air distribution layer can be omitted, i.e. the self-heating patch of the invention can be absent an air distribution layer. In this embodiment, the heat-generating layer can have a textured surface facing the air regulation layer (discussed in more detail below) to facilitate uniform air distribution across its surface.

In one embodiment, the heat-generating layer and/or air distribution layer may be discrete sheets of material. They may alternatively be coatings that are applied directly to an inside surface of the structure by any known method such as screen printing, spray coating, powder coating, etc. Thus, in various embodiments, the invention can comprise:
a heat-generating layer with an air distribution coating;
a heat-generating coating (coated on the internal surface of the bottom barrier layer) with an air distribution coating;
a heat-generating coating (coated on the internal surface of the bottom barrier layer) with no air distribution layer or coating.

In embodiments where no air distribution layer is present, the air regulation layer 24 (discussed in more detail below) is positioned over the outer surface of the bottom barrier layer 12 and the heat-generating layer 20 (see FIG. 2).

Air Regulation Layer

The air regulation layer 24 is continuous over the entire outer surface of the air distribution layer (if present), serving to trap this layer, as well as the underlying heat-generating layer, against the bottom barrier layer. The primary function of the air regulation layer is to provide a controlled level of air permeation via perforations or porosity 25 in the layer, such that the heat-generating layer is limited in its heating rate (and therefore its maximum temperature) by virtue of a restricted supply of oxygen. The use of the air regulation layer affords a more precise means of control of reaction rate than does the adjustment of the reactivity of the heat-generating layer itself: when air entry to the heating layer is rate-limiting, the heating rate may be largely maintained through the consumption of reaction capacity of the heating layer, even though this capacity consumption is accompanied by a reduction in the heating layer's reactivity. Nonetheless, the reactivity of the heat-generating layer can be tailored, e.g., by adjusting the amount of accelerator present, or choice of heat-generating materials, so as not to generate heat too quickly even if exposed directly to air, as a secondary control measure.

As shown in FIGS. 1, 2 and 8, a perimeter seal 30 seals the air regulation layer to the bottom barrier layer around the perimeter of the patch. The seal can be of any suitable type, such as heat seal, ultrasonic seal, radio frequency seal, adhesive seal, or the like, by techniques well known in the art. When used in conjunction with a top barrier layer, the perimeter seal provides an initially hermetic patch 10. When the top barrier layer is peeled away to activate the heat-generating layer, the air regulation layer is positioned to control the ingress of air, and therefore oxygen, into the heat-generating layer, and thus control the degree and duration of resultant heating at the skin surface of the user.

A suitable material for the air regulation layer is a microperforated film, with the density and size of air channels pre-selected, based on such factors as the geometry and construction of the patch, and the amount and nature of the activatable material of the heat-generating layer, to achieve a desired state of heating at the skin surface. The air regulation layer may be a microporous film that has been partially coated or printed to reduce its permeability to a desired level. Examples of microporous films include GORETEX® and CELGARD® films. Another suitable material for the air regulation layer is a needle-perforated plastic film. The number and size of perforations is used to control the air permeation rate.

In one embodiment, the air regulation layer may be altered during use (i.e., after application to the skin) by the addition of needle perforations by the end user. This may be desirable as a means to permit end users more control over the temperature of the patch, given that the addition of more perforations would increase the temperature. It may also serve to ensure that the patch does not overheat before it has been applied to the skin.

Top Barrier Layer

Top barrier layer 26 is continuous over the entire outer surface of the air regulation layer, serving to trap this layer, as well as the underlying heat-generating layer, against the bottom barrier layer. The interface between the top barrier layer 26 and the air regulation layer 24 is a user-separable interface. Prior to use of the self-heating patch 10, the top barrier layer, air regulation layer, and bottom barrier layer are sealed together around the entire periphery of the heat-generating layer to define an oxygen barrier enclosure. Sealing is done as described above for the air regulation layer, and can be done in a single step for both layers. The top barrier layer is present up until the patch is to be used, and its removal will serve to activate the transmission of oxygen through the air regulation layer, and the air distribution layer (if present) to the heat-generating layer, thereby causing heat to be generated.

Any suitable oxygen barrier film of any appropriate thickness can be used for top barrier layer 26. The oxygen barrier material used in top barrier layer 26 can be of any suitable kind, organic or inorganic in nature, including one or more of the polymeric materials identified herein.

Pull Tab

The pull tab 28 can optionally be included as part of the top barrier layer, or as a discrete component attached to the top barrier layer, to facilitate removal of the top barrier layer for activation of the heat reaction. Other methods for initiating removal of the top barrier layer include an overhang of one layer relative to the other. As shown in FIGS. 1 and 2, bottom barrier layer 12, air regulation layer 24, and top barrier layer 26 are coextensive except for the presence of pull tab 28. As an alternative, the top barrier layer can be larger than the bottom barrier layer and/or the air regulation layer, to provide a non-adhered edge region that can be grasped to peel away the top barrier layer. In some embodiments, both the top barrier layer and air regulation layer can be grasped together and pulled away from the bottom barrier layer, and a heat seal at the perimeter between the air regulation layer and the bottom barrier layer may result in a tear-through to a more easily separated (i.e. lower bond strength) interface between the top barrier layer and the air regulation layer, so that the portion of the air regulation layer that includes a heat seal perimeter and the region inward from it is left behind.

In another embodiment, the pull tab may cover a pre-scored area of the top barrier layer. Pulling this tab may expose the pre-scored area and cause one or more tears to propagate from the score outwards toward the perimeter seal, exposing a substantial portion of the underlying air regulation layer without need to disrupt the perimeter seal. An example can be found in U.S. Pat. No. 6,889,483 B2 (Compton et al.), this patent incorporated by reference in its entirety.

Peelable Embodiments

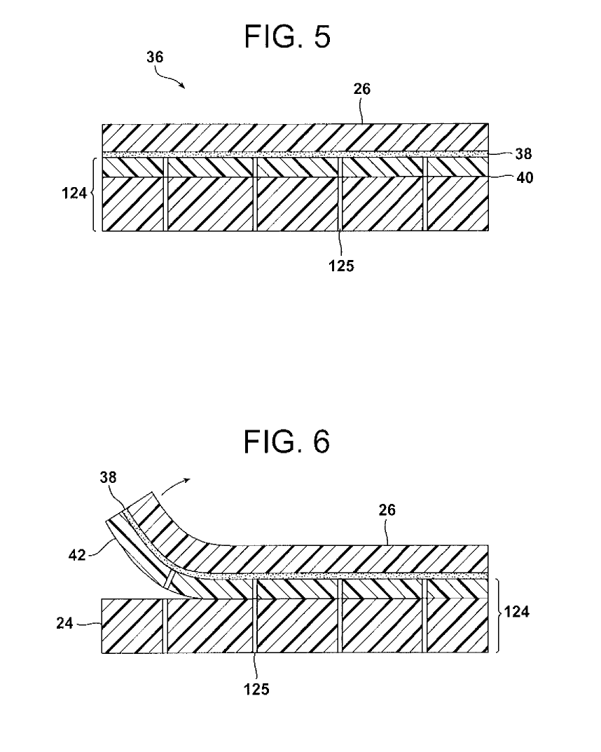

The top barrier layer and the air regulation layer can be two separate films with a relatively weak bond between them, this bond occurring at least within a heat sealed perimeter region, allowing peeling apart. Alternatively, these two layers may be layers of a single peelable composite film structure 36 (see FIG. 5) having an easily separated interface between them. An example of a peelable composite film structure for the top barrier layer and the air regulation layer is a lamination between a barrier film and a multilayer perforated film, where the perforated film has an internal interface with very low bond strength (peelable interface). This embodiment (see FIGS. 5 and 6) makes use of the underlying technology for Sealed Air's LID550P™ film, as disclosed in U.S. Pat. No. 6,033,758 (Kocher et al.), this patent incorporated by reference in its entirety. For the present invention, various aspects of LID550P film may be altered, such as the number and size of perforations, heat shrink properties, layer composition, etc. Example 1, disclosed herein, is an example of a peelable composite film construction useful in the present invention.

### Example 1

### Peelable Composite Film

A. Coextruded Barrier Film

A representative film structure suitable for use as an oxygen barrier film 26 in accordance with the invention is shown in Table 1.

TABLE 1

| | Gauge | Gauge |
|---|---|---|
| Composition | (mils) | (μm) |
| 98% PP1 + 2% AB1 | 0.52 | 13.2 |
| AD1 | 0.17 | 4.4 |
| 80% NY1 + 20% NY2 | 0.12 | 3.0 |
| OB1 | 0.20 | 5.1 |
| 80% NY1 + 20% NY2 | 0.14 | 3.7 |
| AD1 | 0.16 | 4.1 |
| PE1 | 0.40 | 10.1 |
| 98% EM1 + 2% AB2 | 0.29 | 7.3 |

Example 1 as shown has a total thickness of about 2.0 mils, or 50.9 μm.

B. Perforated Sealant Film

A representative film structure suitable for use as a sealant film 124 in accordance with the invention is shown in Table 2.

TABLE 2

| | Gauge | Gauge |
|---|---|---|
| Composition | (mils) | (μm) |
| 96% EV1 + 4% AB3 [layer to be bonded to barrier | 0.06 | 1.4 |
| film] | | |
| 95% (75% PE2 + 25% PE3) + 4% AF1 + 1% AB4 | 0.29 | 7.3 |
| EV2 | 0.11 | 2.9 |
| PP2 | 0.11 | 2.9 |
| 95.5% (50% PE3 + 50% PE4) + 2.64% AF2 + .68% | 0.68 | 17.3 |
| AF3 + .68% AF4 + 0.5% AB5 [layer to be sealed | | |
| to bottom sub-assembly] | | |

Example 2 as shown has a total thickness of about 1.25 mils, or 31.8 μm. The sealant film is perforated. Resins for the above films are identified in Table 3.

TABLE 3

| Material | Tradename Or | |
|---|---|---|
| Code | Designation | Source(s) |
| AB1 | FSU 93E ™ | Schulman |
| AB2 | 10853 ™ | Ampacet |
| AB3 | 101104 ™ | Ampacet |
| AB4 | KAOPOLITE SF ™ | Kaopolite |
| AB5 | ZEEOSPHERE W210 ™ | 3M |
| AD1 | PLEXAR ™ PX2009 ™ | LyondellBasell |
| AF1 | KEMESTER ™ 300 SPECIAL ™ | PMC-Biogenics |
| AF2 | CRF104 ™ | Takemoto Oil and Fat |
| AF3 | WITCONOL 695 ™ | Chemtura |
| AF4 | PATIONIC 907 ™ | Caravan |
| EM1 | SP2205 | Westlake |
| EV1 | EF437AA ™ | Westlake |
| EV2 | ESCORENE LD318.92 ™ | ExxonMobil |
| NY1 | ULTRAMID ™ B40 ™ | BASF |
| NY2 | GRIVORY ™ G21 NATURAL | EMS-Grivory |
| OB1 | SOARNOL ™ ET3803 | Nippon Gohsei |
| PE1 | M6020 ™ | LyondellBasell |
| PE2 | DOW ™2045.04 | Dow |
| PE3 | DOW ™2037 | Dow |
| PE4 | ATTANE ™ 4202 | Dow |
| PP1 | 3571 ™ | Total |
| PP2 | PRO-FAX SR257M ™ | LyondellBasell |

AB1 is an antiblock/slip masterbatch having about 88% low density polyethylene with 9% diatomaceous earth silica and 3% erucamide, each component by weight of the masterbatch.

AB2 is an antiblock masterbatch having about 81%, by weight of the masterbatch, of linear low density polyethylene, and about 19%, by weight of the masterbatch, of an antiblocking agent (diatomaceous earth).

AB3 is an antiblock masterbatch having low density polyethylene with alkali aluminosilicate ceramic spheres.

AB4 is an antiblock made up of anhydrous aluminum silicate.

AB5 is an antiblock made up of alkali aluminosilicate ceramic spheres.

AD1 is a maleic anhydride grafted high density polyethylene that acts as a polymeric adhesive (tie layer material).

AF1 is an antifog agent having a blend of glycerol fatty acid ester and propylene glycol.

AF2 is an antifog agent having a blend of glycerol fatty acid ester and propylene glycol.

A3 is an antifog agent comprising a glycerol fatty acid ester.

A4 is an antifog agent comprising a glycerol fatty acid ester.

EM1 is an ethylene/methyl acrylate copolymer with a methyl acrylate content of about 20% by weight of the copolymer.

EV1 is ethylene/vinyl acetate copolymer with a vinyl acetate content of less than 10% by weight of the copolymer.

EV2 is ethylene/vinyl acetate copolymer with a vinyl acetate content of about 9% by weight of the copolymer.

NY1 is nylon 6 (polycaprolactam).

NY2 is an amorphous copolyamide (6I/6T) derived from hexamethylene diamine, isophthalic acid, and terephthalic acid.

OB1 is an ethylene/vinyl alcohol copolymer (EVOH) with about 38 mole % ethylene.

PE1 is a high density polyethylene homopolymer resin.

PE2 is an ethylene/octene-1 copolymer with a 6.5 weight % octene content, and a density of 0.920 grams/cc.

PE3 is an ethylene/octene-1 copolymer with a 2.5 weight % octene content, and a density of 0.935 grams/cc.

PE4 is a heterogeneous ethylene/octene-1 copolymer with a 9 weight % octene content, and having a density of 0.912 g/cc.

PP1 is a propylene homopolymer.

PP2 is a propylene/ethylene copolymer.

All compositional percentages herein are by weight, unless indicated otherwise.

### Example 2

### Peelable Composite Film

In another embodiment, the peelable composite film structure can be as described in Example 1, but wherein the barrier layer comprises a saran-coated PET. An advantage with this barrier material is that it can be reverse trap printed to provide labeling and instructions visible from the exterior of the patch initially, then no longer present after activation, that is, after the top barrier layer has been peeled away.

In either example, one method of making the peelable composite film is to:

1) provide or produce an oxygen barrier film, e.g. a blown, non-oriented film;

2) provide or produce a sealant film, e.g. a shrinkable sealant film;

3) corona-treat a surface of the barrier film, and corona-treat the surface of the sealant film that will be adhered to the corona-treated surface of the barrier film;

4) advance both the barrier and sealant film between heated rollers such that the corona-treated surfaces face and are brought in contact with one another, to produce the composite film. Thus, in the case of Example 1, the layer of the barrier film comprising the EMA is corona-treated, the layer of the sealant film comprising the EVA is corona-treated, and the two corona-treated layers are bonded one to the other. In one embodiment, a laminating adhesive 38 such as polyurethane can be used to bond the barrier film to the sealant film. After the peelable composite film is incorporated into the self-heating patch, and it is desired to activate the patch, the barrier film and part of the perforated sealant film of the composite can be peeled away, as the composite will cohesively fail at the boundary 42 of the PP2 and sealant layer 24 of the perforated sealant film 124. After peeling, all that is left of the peelable composite film 36 is the sealant layer comprising the PE3 and PE4 materials. This layer includes perforations.

In either example, the perforated film layer that remains behind after delamination (i.e. the air regulation layer) can optionally be pigmented to hide interior components of the patch and blend in with the skin, much as a bandage does. This layer can optionally be colored with a thermochromic ink or pigment, serving to confirm that the patch is heating properly by a visual cue to the wearer.

Method of Assembly

The self-heating patch may be assembled by a variety of methods.

In one embodiment, the final patch structure is assembled by bringing together two continuous webs, top sub-assembly 32 and bottom sub-assembly 34. These two sub-assemblies can each be prepared by a series of steps that modify a starting film web.

For top sub-assembly 32, the starting web 36 can in one embodiment be the peelable (LID550P) structure described herein. A discrete heat-generating layer 20 and discrete air distribution layer 22 can be placed one on top of one another, and the resulting composite can be applied to the peelable composite film 36 on the inner perforated side. A slight degree of thermoforming of film 36 may be used to create recesses for these interior add-on layers.

In one embodiment, a lamination process similar to what is used in applying patches to bag material in making TBG™ barrier bags can be used.

In one embodiment, it can be advantageous to pre-combine the heat-generating and air distribution layers, then bring a continuous web of this two-layer material into a cutting and lamination operation to apply as discrete patches to film 36. In this embodiment, a desirable air distribution layer 22 comprises a nonwoven material that lends tensile strength to the combined web, and also provides suitable lamination surfaces on both its sides. A nonwoven made of a low-melting polymer such as high-vinyl acetate content EVA, used for hot-melt adhesives can be well suited to heat lamination through surface melting. The low-melting nature of the EVA can also provide the fail-safe function as well (the entire nonwoven can melt and seal over the air-access surface of the heat-generating layer).

For the bottom sub-assembly 34, the starting web can be the bottom barrier film 12. Since the components of the bottom sub-assembly 34 will in some embodiments have substantially the same geometry as, and some of the same functional characteristics as an adhesive bandage, a production process based on a method for making adhesive bandages (but not including the step of cutting them apart) may be applicable to its construction.

The final assembly of the self-heating patch of the invention, from top and bottom sub-assemblies 32 and 34, can in one embodiment comprise three steps:

1) adding suitable activation chemicals to the exposed surface of the heat-generating layer 20, transitioning this layer from an air-stable (i.e. oxygen stable) state into an air-reactive (i.e. oxygen reactive) state. This can be done by any suitable means, such as by spraying or flood-coating an aqueous activator solution.

2) bringing the sub-assemblies together so as to fully encapsulate the now-active heat-generating layer 20. This may be done as an adhesive lamination or heat lamination with nip rolls that have recesses to limit the compression to the perimeter region. It may also be done as a packaging operation, treating the top and bottom sub-assemblies as package webs and sealing them together on a MULTIVAC™ thermoforming machine or the like. A perimeter seal 30 is thus made in seal region “A” (see FIGS. 1 and 2), and the package is rendered initially hermetic. While the perimeter seal can be made by using conventional heat sealing techniques, any alternative forms of sealing can be employed, including radio frequency (RF) sealing, ultrasonic sealing, or permanent adhesive.

3) cutting the continuous web of combined subassemblies into individual patches. This step may employ die cutting and/or slitting methods such as are present on current machines available from Multivac, or on commercially available adhesive bandage-making machines.

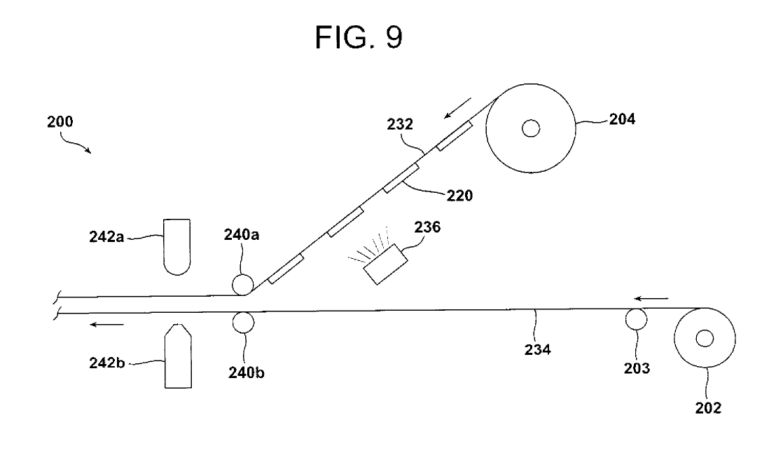

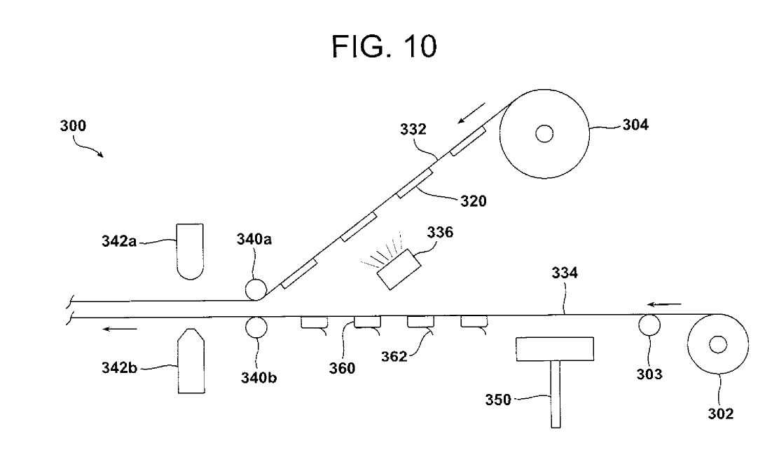

FIGS. 9 and 10 show alternative embodiments of a method of making a self-heating patch. In FIG. 9, apparatus 200 includes bottom feed roll 202 that feeds out a bottom sub-assembly 234 across bottom idler roll 203. Top feed roll 204 feeds out a top sub-assembly 232. At station 236, an activator solution is sprayed or coated onto heat-generating layer 220. The top and bottom sub-assemblies are then brought together at top and bottom nip rolls 240a/240b. Top seal bar 242a and bottom seal anvil 242b seal the sub-assemblies together, creating the perimeter seal for the self-heating patch. FIG. 10 is similar to FIG. 9, apparatus 300 including bottom feed roll 302 that feeds out a bottom sub-assembly 334 across bottom idler roll 303. Top feed roll 304 feeds out a top sub-assembly 332. At station 336, an activator solution is sprayed onto heat-generating layer 320. The top and bottom sub-assemblies are then brought together at top and bottom nip rolls 340a/340b. Top seal bar 342a and bottom seal anvil 342b seal the sub-assemblies together, creating the perimeter seal for the self-heating patch. Forming device 350 creates formed pockets 360 that can be filled with a therapeutic agent such as cosmetics or skin cream. These pockets can be lanced or pre-perforated and covered with a peel tab 362 that can be removed by the end-user just prior to application of the self-heating patch to the skin surface.

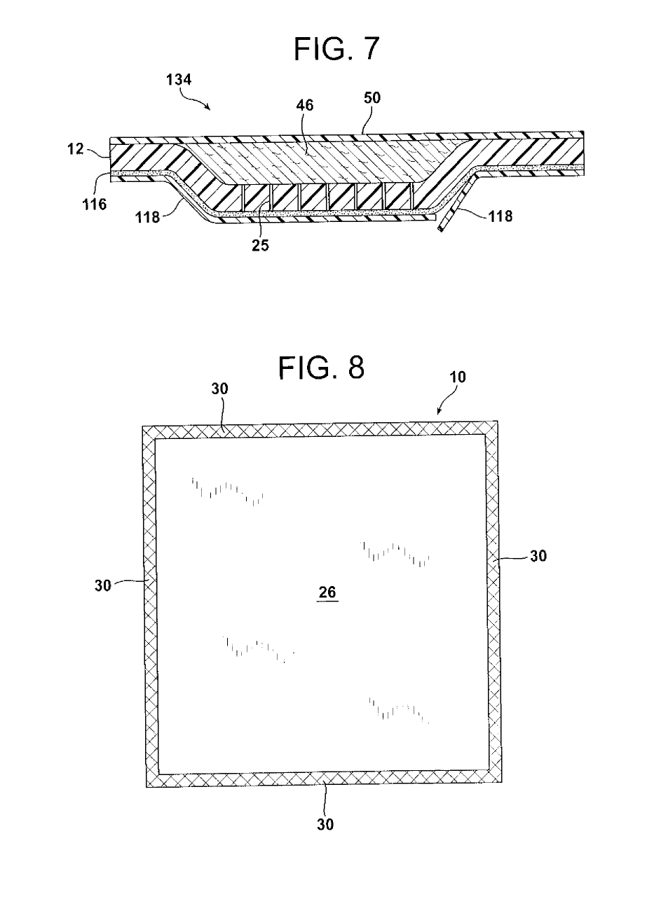

In one embodiment (see FIG. 7), a shallow pocket can be employed in the bottom barrier layer as a means to hold a relatively large amount of a therapeutic material 46, such as a skin treatment material. The top sub-assembly is the same as previously described. The bottom sub-assembly includes a recess in the bottom barrier layer 12 filled with a therapeutic material, and underlying perforations 25 in barrier layer 12 covered by the adhesive liner (the holes are to allow the treatment to be transferred to the skin, and a separator film 50 that covers the sub-assembly so as to maintain separation between the therapeutic material 46 and the heat-generating layer 20 after final assembly. Peelable liner 118 extends across the bottom of the self-heating patch, covering adhesive layer 116.

Heat generating materials contemplated for this invention, which are characterized by high energy density and high rates of reaction with oxygen, are not currently used in dermal patch applications, where a strict upper temperature limit may not be exceeded.

In an alternative embodiment, when a top barrier film is not present in the self-heating patch, a method of assembly similar to the above is followed, but in which the steps disclosed in the second aspect of the invention, found in the Summary, are followed. Thereafter, the self-heating patch is packaged in a barrier pouch to quench the activity of the heat-generating layer.

In another embodiment for making a self-heating patch, a first web can be provided, comprising a bottom sub-assembly comprising a bottom barrier layer and a skin contact layer; an air-activated heat-generating layer is applied to the bottom sub-assembly; an activation agent is applied to the air-activated heat-generating layer; an air regulation layer or a peelable composite (as described herein) is provided as a second web; the first and second webs are brought together to encapsulate the air-activated heat-generating layer; and the first and second webs are cut and sealed to form a plurality of self-heating patches each with a perimeter seal.

Test Performed

A self-heating material manufactured by Rechargeable Battery Corporation (RBC), College Station, Tex., was used to test the ability of a delaminating pre-perforated film for controlling air ingress and thus moderating the temperature rise. A barrier pouch was made of a material consisting of LID550P™ film on one side of the pouch, and T6225B™ film on the other side of the pouch. Both of these materials are available from Cryovac, Inc. The RBC material (branded as COOKPAK™ for MRE's) reacted so quickly upon exposure to air that a sealed package containing the active layer of RBC material in a barrier pouch with a removable layer had to be placed in the fabricated pouch, and the removable film peeled away, then air quickly pressed from the pouch and heat sealed with an impulse seal device. Doing so stopped the exothermic reaction by stopping the flow of oxygen. The LID550P portion was peeled via delamination, to expose the perforations in the film. However, the RBC pack had a film covering with large holes (this being what was exposed by peeling the removable layer) and the LID550P perforations did not match up to the large holes. Thus, a push pin was used to create additional small perforations (total of about 40 tiny holes). The heater pack, which normally reaches a temperature of at least 130° C., reached a maximum temperature of only 52° C. with this technique, demonstrating that utilizing the appropriate number and size of small perforations can control the exothermic reaction by modulating the ingress of air (oxygen). A properly designed perforation pattern can be used to tailor the patch to the desired temperature as well as duration for the heating reaction. The COOKPAK material would typically expire after 30 minutes; however, with the micro-perforations, the reaction was extended to about 3 hours operating in the range of 52 to 46° C.

Temperature-Responsive Mechanism/Fail-Safe Embodiments

Overheating of the air-activated heat-generating layer could cause discomfort or even burns to the wearer of the patch and therefore must be strictly avoided. A desired upper temperature limit is about 43° C. at the skin surface. In some embodiments, in accordance with the invention, a temperature-responsive mechanism can be included in the self-sealing patch to assure temperature control during the useful life of the self-sealing patch.

In one embodiment, this function is achieved by means of a change in shape of the air distribution layer upon heating above a predetermined upper temperature limit of the patch. This change in shape may be enabled by a softening or melting of all or part of the layer, or a component of the layer. A necessary result of this shape change would be to substantially reduce the degree of surface discontinuity on either or both surfaces (i.e., smoothing of the surface or surfaces), and/or a reduction in the porosity of the air distribution layer. “Both surfaces” here refers to the surface of the air distribution layer that is positioned closest to the top of the patch, and to the surface of the air distribution layer closest to the heat-generating layer.

An example of an air distribution layer capable of performing this function would be a wax-coated nonwoven mat. If the mat gets too hot, the wax melts and, by virtue of surface tension, closes off pores and/or reduces surface discontinuities in the layer, substantially reducing air access to the heat-generating layer.

In another embodiment, a cold-embossed perforated film adapted to “de-emboss” itself, i.e. to undergo a reduction in surface discontinuities, upon heating, can be used by the same mechanism whereby a cold-stretched film will shrink upon heating.

In yet another embodiment, a frit-type layer having a wax content can be used.

Alternatively, wax particles can be disposed on the surface of the heat-generating layer facing the air distribution layer. The melting of the wax would close off the pores in the frit, or, in the case of isolated particles, the wax particles can form a continuous layer upon melting.

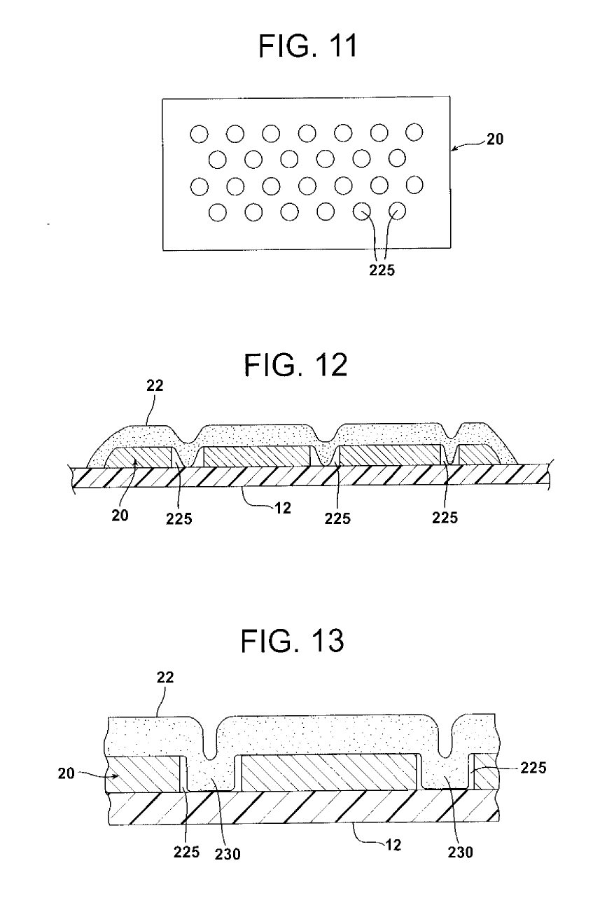

In an alternative embodiment (see FIGS. 11 to 13), a temperature-limiting device can be provided by creating a regular pattern of small holes 225 in the heat-generating layer before applying the heat-generating layer to a top barrier layer (if present) or a barrier bottom layer, and then covering the heat-generating layer with a heat sealable microporous film 22 such as CELGARD™ microporous film. Spot seals 230 are then made between the microporous film and the backside barrier film layer in the hole regions. This device and procedure confines the activatable material of the heat generating layer, and prevents any significant material redistribution resulting from cohesive failures. In one embodiment, the microporous film and/or the bottom barrier film is lightly adhered to the surface of the heat-generating layer by means of a discontinuous adhesive layer that does not significantly impede the air flow to the heater.

One aspect of the microporous film is that it has so many pores so closely spaced that there is effectively no need for lateral air diffusion across the heater surface from a pore site, permitting the entering air to reach substantially all points on the surface of the heat-generating layer. This high pore density allows the microporous film to function effectively in the absence of an air distribution layer.

In another embodiment, the microporous film can have a pattern of low-melting (wax, etc) particles 51 applied or printed onto its outer surface. If a certain pre-determined temperature in the heat-generating layer is reached, these particles melt and flow into the pores of the microporous film to substantially shut off air flow and prevent overheating. Because the microporous film pores are so small, the liquid would be held in by capillary attraction and would not desorb to re-open the pores.

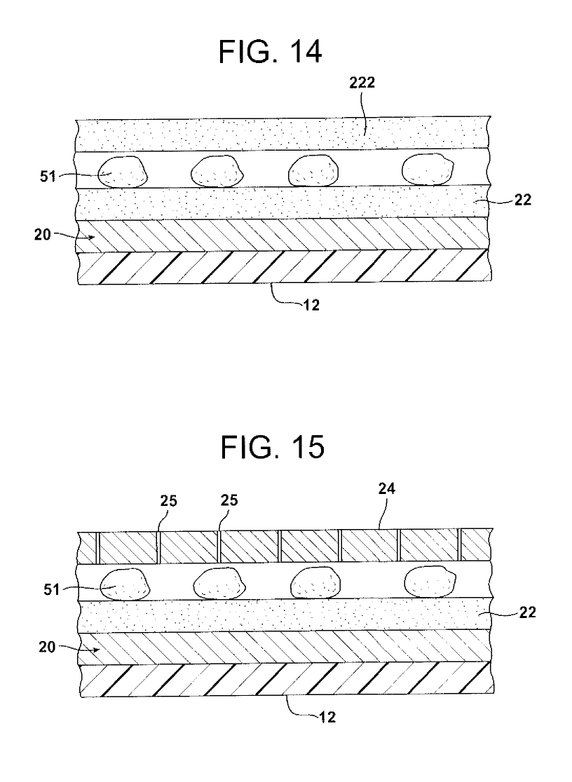

An alternative variant on this approach is to have a low melting particle layer between two porous films. The outer porous film 222 could be another microporous film (see FIG. 14) or the inner layer 24 of a peelable composite (see FIG. 15), e.g. LID 550P or a similar composite as discussed hereinabove, i.e., the outer porous film can be the air regulation layer as defined herein.

A method of making a self-heating patch, using a microporous film, low melting point material such as wax, and a peelable composite, is as follows:
1) spray-coat wax particles onto the inner surface of a peelable composite,
2) add a microporous film to the spray-coated side of the peelable composite,
3) lightly adhere the microporous film to the peelable composite, such that there is at least significant open space around the wax particles between the layers,
4) add a perforated electrolyte-free heat-generating layer to a continuous barrier bottom layer,
5) add electrolyte solution to the heat-generating layer without contaminating the heat sealable surfaces of the barrier film exposed around the heat-generating layer,
6) bring the top sub-assembly formed by steps 1) to 3), with the microporous film down, over the top of the bottom sub-assembly formed by the electrolyte-containing heat-generating layer and the barrier bottom layer,
7) make spot seals in the holes of the heat-generating layer, and a perimeter seal, to fully trap the heat-generating layer and define airtight enclosures, and
8) apply any additional elements, if present, in a separate process or processes (e.g. adhesive film or coating, skin contact pad, skin cream, adhesive release liners, pull tab for peelable web) and,
9) cut apart the top and bottom sub-assemblies into individual patches.

Spray-coating could in one embodiment be done with two or more wax types having different melting points, to give a graded shutoff valve effect. At each melting temperature, an increasing percentage of pores would be closed off.

### Examples

### Self-Heating Patch

Film Sample Preparation

Samples of CELGARD™ 2325 tri-layer PP(polypropylene)/PE (polyethylene)/PP microporous film were either used as-is or pre-treated with wax. The wax pre-treatment consisted of spray coating molten wax onto one side of the film using a CHAMP™ 10S LCD spray gun (Glue Machinery Corporation). The wax spray was applied so as to cover only a fraction of the film surface with particles of a width of from 5 to 200 microns. The appearance of the film remained opaque white, indicating that the wax had not substantially entered the voids in the film. If the film was subsequently heated above the melting point of the wax, it became transparent as a result of the molten wax filling the voids in the film.

Specific waxes used in the examples are:

Wax A: Melting point 129° F. paraffin wax.
Wax B: Melting point 160° F. paraffin wax
Wax C: Blend of 1 part Wax A with 2 parts Wax B.
Wax D: Wax C with blue tint additive for visualization

Specific wax-coated films used in the examples are identified as follows:

FS-1: CELGARD™ 2325 control
FS-2: CELGARD™ 2325 spray-coated with Wax A (approx. 50% coverage)
FS-3: CELGARD™ 2325 spray-coated with Wax C (approx. 50% coverage)
FS-4: CELGARD™ 2325 spray-coated with Wax D (approx. 85% coverage)

Self-Heating Sample Preparation

The self-heating material used was obtained from RBC Technologies as their COOKPAK MRE heater assemblies, which contained a heat-generating layer and various other film layers. For the present examples, only the heat generating layer was employed. The chemistry of the layer is as disclosed in U.S. Pat. No. 7,722,782 (Coffey et al.), this patent incorporated herein by reference in its entirety. It was removed from the COOKPAK assembly inside a nitrogen-filled glove box and used in the construction of temperature-regulated self-heating patch test assemblies for heating rate measurement. The sample preparation is described further in the examples.

### Example 1

A Gurley Densitometer, Model 4150N equipped with a 4320 Automatic Digital Timer, was used to characterize the relative permeation rate of film samples. Pressure was 6.52 psi, sample area was 1 sq. in., and displacement volume was 10 cc. This test method measures the time for a known air volume to pass through a porous film sample under a known air pressure gradient. A longer time value thus indicates a lower permeation rate.

Table 4 below shows the results obtained:

TABLE 4
Air permeation measurements on film samples

| Film Sample | | Gurley Reading |
|---|---|---|
| Designation | Film Sample Description | (seconds +/− std. dev.) |
| 1 | FS-1, as made | 22.4 +/− 0.5 |
| 2 | FS-1, after heating @ | 22.7 +/− 0.2 |
| | 140° F. for 1 minute | |
| 3 | FS-1, after heating @ | 65.8 +/− 0.5 |
| | 275° F. for 1-2 minutes | |
| 4 | FS-1, after heating @ | 245 +/− 3 |
| | 275° F. for 1-2 minutes, then @ | |
| | 282° F. for 1-2 minutes | |
| 5 | FS-2, as made | 42.3 +/− 4.1 |
| 6 | FS-2, after heating @ | 994 (single reading) |
| | 140° F. for 1 minute | |
| 7 | FS-3, as made | 51.4 +/− 13.1 |
| 8 | FS-4, as made | 122 +/− 16 |

The results of Table 4 show the effect of wax coverage in reducing the permeation rate of the microporous film. Sample FS-4 had a heavier coating than did Sample FS-2, and this is reflected in the much lower permeation rate (larger Gurley reading on sample 8 vs. sample 5). The effect of heating above the melting point of the wax is shown for sample FS-2. The permeation rate drops to an almost immeasurably low value after heating at 140° F. for one minute (compare sample 6 with sample 5). During sample preparation, it was noted that the appearance of sample FS-2 changed from white to transparent after heating, which is believed to be a result of substantial filling of the pores with molten wax. This effect is not seen with the control film FS-1 receiving the same heat history (sample 2).

Sample FS-1 is a three-layer microporous film having a lower-melting PE core layer. While the air permeation if this film is not affected by modest heat treatment (sample 2 vs. sample 1), it was seen that higher temperature treatment, near the melting point of polyethylene, will lower the air permeation rate significantly (samples 3 and 4). These results suggest, in an alternative embodiment, an advantageous use of heat-treated three-layer microporous film where a lower air permeation rate is desired.

### Example 2

An Omega Model HH309A four-channel data logger thermometer was used for collection of time-temperature measurements from the surface of test patches constructed using Sample FS-1 to Sample FS-4 films, as described below.

The general procedure for making test patches was to affix a piece of 3″ wide clear packaging tape to the face of a flat rectangular metal frame having a 2″×6″ opening and made from ⅛″ aluminum. Inside a nitrogen-filled glove box, an approximately 1″×2″ test strip of self-heating material was removed from a COOKPAK heater. This sheet, about 1/16″ thick, was affixed to the tape surface centered inside the metal frame so as to leave at least ½″ open margin around all sides. One or two microporous film samples was/were laid over this heating material, waxed side up (where applicable) so as to extend at least ½″ beyond the self-heating material edges. The film sample was press-adhered to the tape surface so as to form a seal around all edges. In some cases a second piece of test film was applied, extending at least ¼″ beyond all of the edges of the first piece, so that it was independently press-sealed to the tape surface around the entire perimeter. The metal frame with test patch assembly was temporarily covered over with a lid to prevent air access, then removed from the glove box. Two thermocouple wires were taped to the underside (tape side) of the test patch, about 1″ apart and centered under the heating material. Temperature monitoring was initiated, then the lid on the metal frame was removed to initiate air access and resulting heating of the test patch. The frame was propped at an angle so that air could access all sides throughout the test.

Table 5 below identifies the test patches constructed.

TABLE 5
Test patch constructions

| Test Patch Designation | Sample Film Description |
|---|---|
| TP-1 (Control) | One layer: FS-1 |
| TP-2 (Control) | Two layers: FS-1 |
| TP-3 | Two layers: FS-3 |
| TP-4 | One layer: FS-4 |

Table 6 below gives summary values associated with the time-temperature profiles measured for each of the test patch samples. All data are derived from the average of two readings from side-by-side thermocouples on each test patch.

TABLE 6
Test patch time-temperature data summaries

| | | Plateau | Heat Duration | |
|---|---|---|---|---|
| Test | T^{max} | range | (min. above | |
| Patch | (° C.) | (° C.) | 35° C.) | Wax appearance after test |
| TP-1 | 136 | none | 14 | N/A |
| TP-2 | 108 | 45-50 | 85 | N/A |
| TP-3 | 63 | 40-45 | 68 | Partially melted in center, |
| | | | | completely melted at edges |
| | | | | (outer layer); completely |
| | | | | melted (inner layer) |
| TP-4 | 57 | 37-42 | 217 | Partially melted in center, |
| | | | | completely melted at edges |

The data in Table 6 for controls (TP-1 and TP-2) show that one or two layers of the microporous film with no wax coating gave insufficient regulation of the maximum temperature, even though the TP-2 sample had enough permeation resistance to eventually regulate the temperature in the 45 to 50° F. range as indicated by the plateau value and extended duration of heating.

The data in Table 6 for TP-3, made using two layers of the lightly wax-coated microporous film, show a maximum temperature limited to 63° C., while affording a plateau of 40 to 45° C. with an approximate heating duration of 68 minutes. As noted, the wax was not completely melted in both film layers of this test patch. When compared with the analogous two-layer control sample TP-2, it is apparent that the wax coating on the two layers in TP-3 served more to reduce the peak temperature than to reduce the plateau temperature, which is a desirable result.

The data in Table 6 for TP-4, made using one layer of more heavily wax-coated film, showed the lowest peak temperature and longest heating duration. As noted, the wax coating on the sample was not completely melted.

### Example 3

### Prophetic Example

A perimeter-sealed self heating patch is constructed from a self heating layer, a top film layer and a bottom film layer, where the self heating layer does not extend to the edges and is sealed between the top and bottom film layers, which come into contact around the edges.

The layers are defined further as follows:

Top Film Layer

A continuous microporous flexible film having average pore size of less than 10 microns, e.g. less than 1 micron, and an air permeation rate that leads to a time of less than about 2000 seconds, and greater than about 200 seconds, when the film is tested in a Gurley tester at 6.52 psi, 10 cc displacement, 1 sq. inch area.

In one embodiment, this film is made from a heat-treated composite microporous film having core layer and skin layers, where the core layer has a lower melting temperature range than the skin layers and the heat treatment is within the melting temperature range of the core layer. In another embodiment, this film is made by printing on the surface of a microporous film.

In another embodiment, this microporous film is peelably laminated to a continuous cover film having an air permeation rate no more than 1/10^{th} the air permeation rate of the microporous film, e.g. no more than 1/100^{th} the air permeation rate of the microporous film.

Bottom Film Layer

A continuous flexible film having an air permeation rate no more than 1/10^{th} the air permeation rate of the top film layer, e.g. no more than 1/100^{th} the air permeation rate of the top film layer, where the air permeation rate of the top film layer is taken as the permeation rate after removal of a peelably laminated cover film from the top film layer if such is present.

Self Heating Layer

A porous composition comprising a mixture of particles of

a) oxidizable metal powder (e.g., Zn, Fe, Al),

b) carbon powder, and

c) wax powder,

and further comprising an aqueous salt solution.

The self-heating patch is constructed so that the top film layer's peelable cover film, if present, is oriented toward the exterior of the top film, i.e. the side that does not face the self-heating layer. The bottom film layer in one embodiment have an adhesive coating over all or part of its exterior surface to facilitate peelable adhesion to the skin. The bottom film layer may optionally be adhered by its exterior surface to additional layers, such as nonwoven material impregnated with a skin treatment agent, and/or adhesive release liners.

In use, the self-heating patch is placed on the skin, and then the peelable cover film (if present) is removed from the top film layer. Air permeates the top film layer and undergoes an exothermic chemical reaction within the self heating layer to oxidize the metal particles. The rate of air permeation serves to control the rate of this chemical reaction and thus the rate of heat-generation, affording a safe level of heating, below the melting temperature range of the wax particles in the self heating layer. As the reaction progresses, the rate of reaction is slower and more constant over time than it would be absent the top film.

If a breach of the enclosure formed by the top and bottom film layers allows air to penetrate at a rate faster than intended, the self heating material may become hotter than intended, and may attain a temperature that is within or above the melting temperature range of the wax particles in the self heating layer, still lower than a temperature at which skin burns could result. This causes the wax particles to become substantially liquid, reducing the porosity of the self heating layer and/or coating air-reactive sites within the layer, and thus restricting air access to the interior of the layer. This serves to reduce the reaction rate and curtail further overheating of the self heating layer, keeping it below the temperature at which skin burns could result.

### Example 4

A perimeter-sealed self heating test patch was constructed as described in Example 2, but with the cover film comprising a single layer of unperforated Sealed Air CT-301™ film. CT-301 is a 30 gauge polyolefin shrink film having a published OTR of 17,000 cc/m2/day/atm.

The test patch showed no temperature rise, confirming that even a very high OTR film, if not perforated, had insufficient air permeation to serve as the air regulation layer. Over a period of about 10 minutes, a series of 22 randomly-placed needle perforations was made in the surface of the CT-301 film that was directly over the heating material, which was 1 inch×1.5 inch in size. It was found that incremental addition of perforations over a 10 minute time period moved the temperature of the patch up from 25° C. to a targeted value, in this case 60-65° C. Afterwards, the temperature declined gradually, such that a temperature of >40° C. was sustained for approximately one hour. This result demonstrates that a useful areal density of needle perforations in a film might be about 15 perforations per square inch, assuming that 40-65° C. is a useful temperature range.

### Example 4A

The experiment of Example 4 was repeated, with the only changes being

a) the addition of a nonwoven material layer, serving as an air distribution layer, between the CT-301 cover film and the heating material. This material was the air distribution layer in the COOKPAK™ material; and
b) the creation of the 22 holes in the CT-301 within a shorter time period, less than one minute.

It was found that, in comparison with Example 4, the peak temperature was higher (approx. 88° C.) and the heating duration was reduced. This result is attributed to the improvement in air flow afforded by the air distribution layer, which in turn led to more heat generation in the sample. The temperature profile was smoother than that of Example 4, presumably a result of the air distribution layer guarding against transient localized air flow blockages and/or uneven oxidation reaction rates due to variable air access across the surface of the heater layer.

### Example 5

A perimeter-sealed self heating test patch was constructed as described in Example 2, but including an air distribution layer and with the cover film being a single layer of CELGARD™ 2325 film which had been lightly coated with RUST-O-LEUM™ spray paint such that a Gurley reading (as defined in Example 1) on a section of the film was 1,118.1 seconds. It was found that the peak temperature was about 72° C., and a 55-72° C. temperature plateau was sustained for about 50 minutes. A sharp temperature fall thereafter indicates that the desired heating rate was sustained until the heating material was almost fully oxidized. This result indicates that a microporous cover film having Gurley reading of approximately 1,100 seconds is useful for achieving a plateau of approximately 65° C. over an extended time period. It is to be expected that, in contact with skin, the resulting increase in heat removal rate would serve to lower the temperature plateau to a safer and more comfortable range, say, 40-55° C. Also, in a self-heating patch of the invention, the thermal insulation effect of intervening layers (e.g., skin contact layer) could serve to reduce the rise in skin temperature to significantly below that of the heater surface.

The present application is directed in various embodiments to the subject matter described in the following paragraphs. These are optional embodiments of any of the first, second, third, fourth, fifth, or any subsequent aspects of the invention as described hereinabove in the Summary of the Invention, and for each aspect, these features can be included alone or in any suitable combination of these features:
wherein a top barrier layer is present, the air-activated heat-generating layer is encapsulated by the top and bottom barrier layers until the top barrier layer is removed from the patch.
an air distribution layer is disposed between the air-activated heat-generating layer and the air regulation layer.
the top barrier layer, if present, is peelably removable from the self-heating patch.
an adhesive layer is disposed adjacent the skin contact layer.
a pull tab is adapted to remove the top barrier layer, if present, to activate the self-heating patch.
a therapeutic agent is disposed in the patch between the bottom barrier layer and the air-activated heat-generating layer.
a separating layer is disposed between the air-activated heat-generating layer and the bottom barrier layer, defining a pocket for holding a therapeutic agent.
a temperature-responsive mechanism is disposed a) within the air-activated heat-generating layer, b) on the air distribution layer, if present, and/or c) on the air regulation layer; and the temperature-responsive mechanism is adapted to melt above a predetermined temperature set point and thereby reduce oxygen intake into the self-heating patch.
a temperature-responsive mechanism, disposed within the air-activated heat-generating layer, comprises wax particles.
the air regulation layer comprises a microporous film or a perforated film.
an outer barrier package enclosing the patch.
at least one perforation is added to the air regulation layer after exposing the air-activated heat-generating layer to air.

Alternative Method

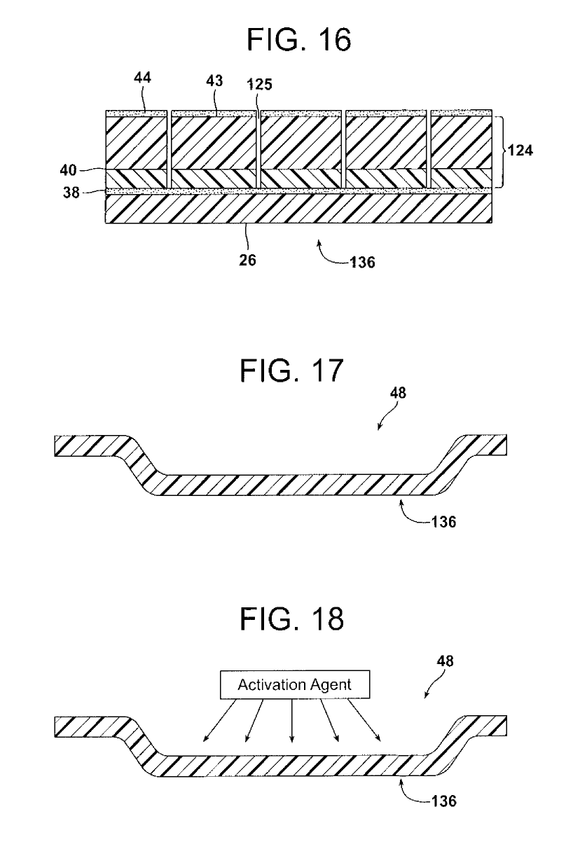

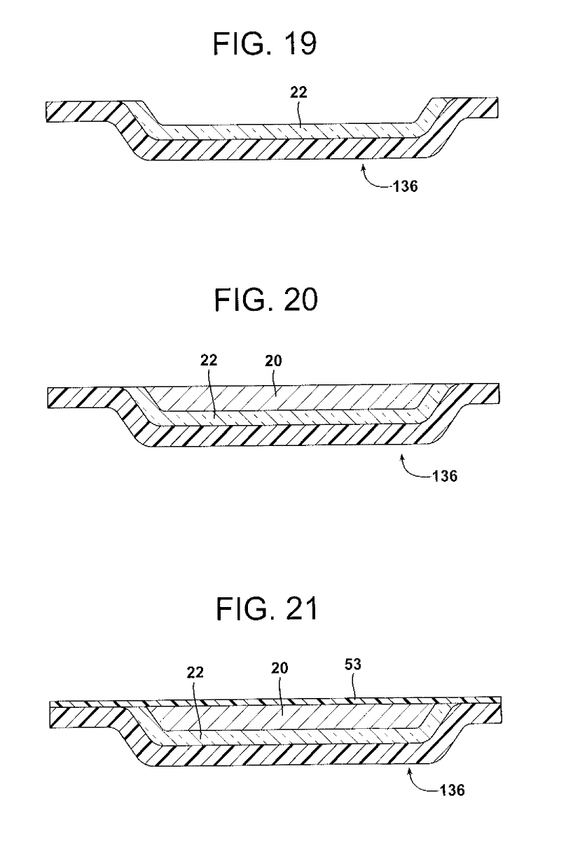

In another embodiment (see FIGS. 16 to 21), a process for making a self-heating patch involves assembling the patch “upside-down” on a thermoforming machine, such as of the type manufactured by Multivac.

This alternative method includes the steps of:

1) providing a peelable composite 136 including a barrier layer 26 and an air regulation layer 124 in the form of a perforated peelable film. In one embodiment, this peelable composite includes a perforated component 124, an unperforated component 26, and a peelable functionality, whereby the unperforated component 26, and optionally a portion of the perforated component 124, can be peeled away at peelable interface 40 to provide a perforated substrate that functions as the air regulation layer. Both the perforated and unperforated components in one embodiment comprise polymeric materials.

An example is the LID550P™ laminate described herein. Other alternatives include analogs of LID550P™ that may have various aspects of LID550P film that can be altered, such as the number and size of perforations, heat shrink properties, layer composition, etc. For example, the peelable composite can comprise a non-heat shrinkable perforated component, and a non-heat shrinkable unperforated component.

In one embodiment, the peelable composite can be reverse printed, i.e. printed on its perforated surface (i.e. the surface that comprises a surface of the air regulation layer), with a suitable skin-tone color or other graphic. This feature masks the air-activated heat generating layer from view. The print layer, if present, can optionally be coated with a material such as an ethylene copolymer or ionomer dispersion that promotes heat sealing.

2) thermoforming the peelable composite 136 (see FIG. 17, showing peelable composite 136 without the details of FIG. 16), with the printed surface (if present), facing up, i.e. towards the interior of a package to be made from the process, to form a thermoformed peelable composite having a shallow pocket 48.

3) disposing an activation agent, if needed, in the shallow pocket 48. This would put the activation agent on a surface of the air regulation layer.

An air distribution layer 22, as described hereinabove, can in one embodiment be included, and disposed on the (optionally printed) surface of the air regulation layer. In this embodiment, an activation agent, if needed, is disposed on the air distribution layer.

4) disposing an air-activated heat-generating layer in the shallow pocket 48, on the surface of the air regulation layer 124, or if present, the air distribution layer 22 (see FIG. 20). If an activation agent is present, the air-activated heat-generating layer 20 is disposed on the activation agent, resulting in conversion of the air-activated heat-generating layer from an air-stable state to an air-reactive state.

Placement of the air distribution layer 22, if present, and the air-activated heat-generating layer 20 into the shallow pocket 48 formed by the thermoformed peelable composite can be done in any suitable way, e.g. manually, or mechanically, e.g. by a robotic pick-and-place process.

The peelable composite 136 and air-activated heat-generating layer 20, together with any (if present) of print layer, heat sealable coating, air distribution layer, and activation agent, comprise a first segment.

5) applying an interface film 53 to the first segment to encapsulate the air-activated heat-generating layer 20 between the first segment and the interface film 53 (see FIG. 21). The interface film 53 can be of any suitable composition, such as that shown for bottom barrier layer 12 of FIGS. 1 and 2;

6) sealing the first and second segments together, along the perimeter of each of the first and second segments, to form a self-heating patch with a perimeter seal.

The sealing process may in one embodiment include vacuumization and/or gas flush steps if so desired. The heat sealing can be accomplished by any suitable type of seal such as a heat seal, ultrasonic seal, radio frequency seal, adhesive seal, or the like, e.g. via a hot plate or a patterned seal bar.

In practice, the respective materials as described above can be advanced in a thermoforming process, with periodic and controlled cutting and sealing of the materials forming the first and second segments to define a plurality of self-heating patches.

The final product as shown in FIG. 21 can be used as a self-heating patch, wherein the peelable component of the peelable composite can be peeled away to expose the air regulation layer 24 (see FIG. 6), and the interface film 53 can be applied to the skin. Alternatively, the final product of the alternative method, i.e. the self-heating patch of FIG. 21, can be combined with a skin-contacting bottom sub-assembly 134 such as that shown in FIG. 7. In this alternative, interface film 53 is adhered to separating film 50 to provide a self-heating patch in which a shallow pocket is employed in the bottom barrier layer as a means to hold a relatively large amount of a therapeutic material 46, and underlying perforations 25 allow the therapeutic material to be transferred to the skin. Alternatively, the self-heating patch of FIG. 21 can be combined with a skin-contacting bottom sub-assembly such as 34 shown in FIG. 4. In this alternative, interface film 53 is adhered to the bottom barrier film 12.

What is claimed is:

1. A flexible, self-heating patch comprising:
a) a bottom barrier layer;
b) an air-activated heat-generating layer; and
c) an air regulation layer;
wherein the air-activated heat-generating layer is encapsulated by the air regulation layer and the bottom barrier layer;
wherein the temperature of the patch, when the air-activated heat-generating layer has been exposed to air, is controlled at least in part by the air regulation layer; and
wherein a perimeter seal seals the air regulation layer to the bottom barrier layer around the perimeter of the patch.

2. The self-heating patch of claim 1 further comprising a top barrier layer disposed on the air regulation layer.

3. The self-heating patch of claim 2 wherein the top barrier layer is peelably removable from the self-heating patch.

4. The self-heating patch of claim 1 further comprising an air distribution layer disposed between the air-activated heat-generating layer and the air regulation layer.

5. The self-heating patch of claim 1 further comprising a skin contact layer disposed on an outer surface of the bottom barrier layer.

6. The self-heating patch of claim 5 wherein a therapeutic agent is disposed in the skin contact layer.

7. The self-heating patch of claim 1 wherein a temperature-responsive mechanism is disposed within the self-heating patch, adapted to prevent overheating of the self-heating patch by means of a change in phase or a change in shape.

8. A method of making a plurality of self-heating patches comprising:
a) providing an air regulation layer;
b) applying an air-activated heat-generating layer to the air regulation layer to form a top sub-assembly as a first web;
c) providing a bottom sub-assembly comprising a bottom barrier layer and a skin contact layer to form a second web;
d) applying an activation agent to the air-activated heat-generating layer to change the air-activated heat-generating layer from an air-stable state to an air-reactive state;
e) bringing the first and second webs together to encapsulate the air-activated heat-generating layer; and
f) cutting and sealing the first and second webs to form a plurality of self-heating patches each with a perimeter seal.

9. The method of claim 8 further comprising placing each of the plurality of self-heating patches in a barrier pouch.

10. The method of claim 8 further comprising an air distribution layer disposed between the air-activated heat-generating layer and the air regulation layer.

11. The method of claim 8 wherein an adhesive layer is disposed adjacent the skin contact layer.

12. The method of claim 8 wherein a therapeutic agent is disposed in the skin contact layer.

13. The method of claim 8 wherein a temperature-responsive mechanism is disposed within the self-heating patch to prevent overheating of the self-heating patch by means of a change in phase or a change in shape.

14. The method of claim 13 wherein the temperature-responsive mechanism comprises a wax coat disposed on the air regulation layer and adapted to melt above a predetermined temperature and thereby reduce oxygen intake into the heat-generating layer.

15. A method of making a plurality of self-heating patches comprising:
a) providing a peelable composite comprising a top barrier layer and an air regulation layer;
b) applying an air-activated heat-generating layer to the air regulation layer to form a top sub-assembly as a first web;
c) providing a bottom sub-assembly comprising a bottom barrier layer and a skin contact layer to form a second web;
d) applying an activation agent to the air-activated heat-generating layer to change the air-activated heat-generating layer from an air-stable state to an air-reactive state;
e) bringing the first and second webs together to encapsulate the air-activated heat-generating layer, and
f) cutting and sealing the first and second webs to form a plurality of self-heating patches each with a perimeter seal.

16. The method of claim 15 further comprising an air distribution layer disposed between the air-activated heat-generating layer and the air regulation layer.

17. The method of claim 15 wherein the top barrier layer is peelably removable from the self-heating patch.

18. The method of claim 15 wherein an adhesive layer is disposed adjacent the skin contact layer.

19. The method of claim 15 wherein a therapeutic agent is disposed within the skin-contact layer.

20. The method of claim 15 wherein a temperature-responsive mechanism is disposed within the self-heating patch, adapted to prevent overheating of the self-heating patch by means of a change in phase or a change in shape.

21. A flexible, self-heating patch comprises:
a) a first segment comprising
i) a thermoformed peelable composite comprising a barrier layer and an air regulation layer; and
ii) an air-activated heat-generating layer; and
b) a second segment comprising an interface film;
wherein the air-activated heat-generating layer is encapsulated by the air regulation layer and the interface film;
wherein the temperature of the patch, when the air-activated heat-generating layer has been exposed to air, is controlled at least in part by the air regulation layer; and
wherein a perimeter seal seals the first segment to the second segment around the perimeter of the patch.

22. The flexible, self-heating patch of claim 21 further comprising an air distribution layer disposed between the air-activated heat-generating layer and the air regulation layer.

23. The flexible, self-heating patch of claim 21 further comprising an activation agent disposed on the air regulation layer.

24. The flexible, self-heating patch of claim 22 further comprising an activation agent disposed on the air distribution layer.

25. A method of making a self-heating patch comprises
a) providing a peelable composite comprising a barrier layer and an air regulation layer;
b) thermoforming the peelable composite to form a pocket;
c) applying an activation agent to the pocket;
d) applying an air-activated heat-generating layer to the pocket;
wherein the peelable composite, activation agent, and air-activated heat-generating layer comprise a first segment;
e) applying an interface film to the first segment to encapsulate the air-activated heat-generating layer between the first segment and the interface film; and
f) sealing the perimeter of the first and second segments to form a self-heating patch with a perimeter seal.

26. The method of claim 25 further comprising an air distribution layer disposed between the air-activated heat-generating layer and the air regulation layer.

27. The flexible, self-heating patch of claim 25 wherein the activation agent is disposed on the air regulation layer.

28. The flexible, self-heating patch of claim 26 wherein the activation agent is disposed on the air distribution layer.
